(19) United States
(12) Patent Application Publication
(10) Pub. No.: US 2011/0295357 A1
(43) Pub. Date: Dec. 1, 2011

(54) Abdominal Aortic Stent

(76) Inventors: Po-Jen Ko, Taipei City (TW); Ching-Yang Wu, Linkou Township (TW); Yun-Hen Liu, Guishan Shiang (TW); Hui-Ping Liu, Taipei City (TW)

(21) Appl. No.: 12/790,956

(22) Filed: May 31, 2010

(51) Int. Cl. A61F 2/82

(52) U.S. Cl. 623/1.15

(57) ABSTRACT

An abdominal aortic stent includes a first sub-stent and a second sub-stent, each having a front end and the circumference of the front end of each sub-stent is half of the circumference of the abdominal aorta, and when the circumference is gradually reduced to a rear end of each sub-stent, it has entered one side of the bilateral femoral arteries, wherein the rear ends of the sub-stents with full circumference of the cross-sectional area of the femoral artery are included therein; the first and second sub-stents are coated with external removable membrane to compress the sub-stents to generate smaller circumferences, and when the membrane has been removed, the sub-stents are fully extended to reconstruct the vascular flow path.

### BACKGROUND OF THE INVENTION

1. Field of the Invention

This invention relates to an abdominal aortic stent. More specifically, this invention relates to an abdominal aortic stent structure which can be easily implanted to reconstruct a vascular flow path.

2. Description of Related Art

The development of stents began since the late '90s to reconstruct the normal vascular flow path by inserting the stents to separate the aneurysm. It is so called the endovascular aneurysm repair (EVAR).

Conventionally, in clinical endovascular aneurysm repair practice, a metal wire is inserted from both sides of the femoral artery, and an appropriate stent size would be determined by surgery angiography according to the location, length and influential area of the aneurysm. The stent is then guided to an expected destination through the metal wire. After the membrane wrapped outside the stent is removed, the stent can fully extend to reconstruct vascular flow path. According to the clinical study, the endovascular aneurysm, comparing with the conventional surgery, has the following advantages of: (1) shorter operative time, (2) small amount of blood loss, and (3) shorter recovery time.

Referring to FIGS. 1 to 3, the stent surgery would only generate a small wound, which may not damage the tissues near blood vessels. Also, the stent surgery only causes little blood loss. Thus, the stent surgery has become a trend of modern vascular surgery. On the contrary, conventional abdominal aortic aneurysm surgery is a major vascular surgery, which would generate a 30 to 40 cm abdominal incision from the xiphoid (below chest) to the top part of the pubic bone. The stent surgery can be done by using a metal wire (1), a membrane (3) and a main stent structure (2). With the assistance of X-ray fluoroscopy, the stent can be sent from both sides of the femoral artery into the dilatation position of the abdominal aorta. After the main stent (2) is well positioned, the membrane (3) will be pulled down to have the main stent (2) deploy within the abdominal aortic aneurysm, such that the blood can flow into the main stent (2). If continuously pulling down the membrane (3), the main stent (2) will be completely open. Likewise, to connect the main stent (2) to a sub-stent (5), the first step is to put another metal wire (4) through a branch opening (2a) of the main stent (2), and the sub-stent (5) can be guided along the metal wire (4) to an appropriate location. However, sometimes if the blood vessel of the patient has a larger angle, or the branch opening (2a) is hard to be located, it may be difficult to cannulate the metal wire (4) to the branch opening (2a) of the main stent (2), which may increase the surgery time and even lead to surgery-related complications.

Therefore, there remains a need for a new and improved stent structure to overcome the abovementioned issues to effectively implant the stent into the abdominal aorta to reconstruct vascular flow path.

### SUMMARY OF THE INVENTION

It is an object of the present invention to provide a stent structure to effectively implant the stent into the abdominal aorta to reconstruct vascular flow path.

It is another object of the present invention to provide a stent structure with two sub-stents to shorten the surgery time, reduce the surgery-related complications and help the patient recover faster.

It is a further object of the present invention to apply a stent structure (having two sub-stents) in renal artery, wherein small stents used to connect the renal artery can be placed at a first space and a second space preserved in the appropriate place in the sub-stents.

The present invention together with the above and other advantages may best be understood from the following detailed description of the embodiments of the invention illustrated in the drawings below.

### BRIEF DESCRIPTION OF THE DRAWINGS

FIG. 1 illustrates a prior art, related to a conventional abdominal aortic stent.

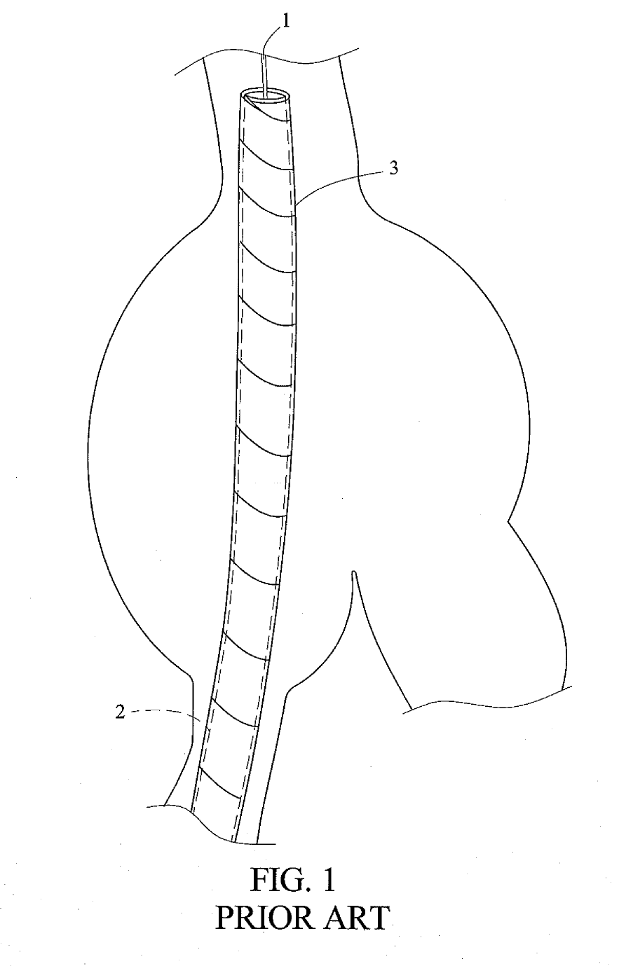

FIG. 2 illustrates a prior art, related to a conventional abdominal aortic stent with a branch opening.

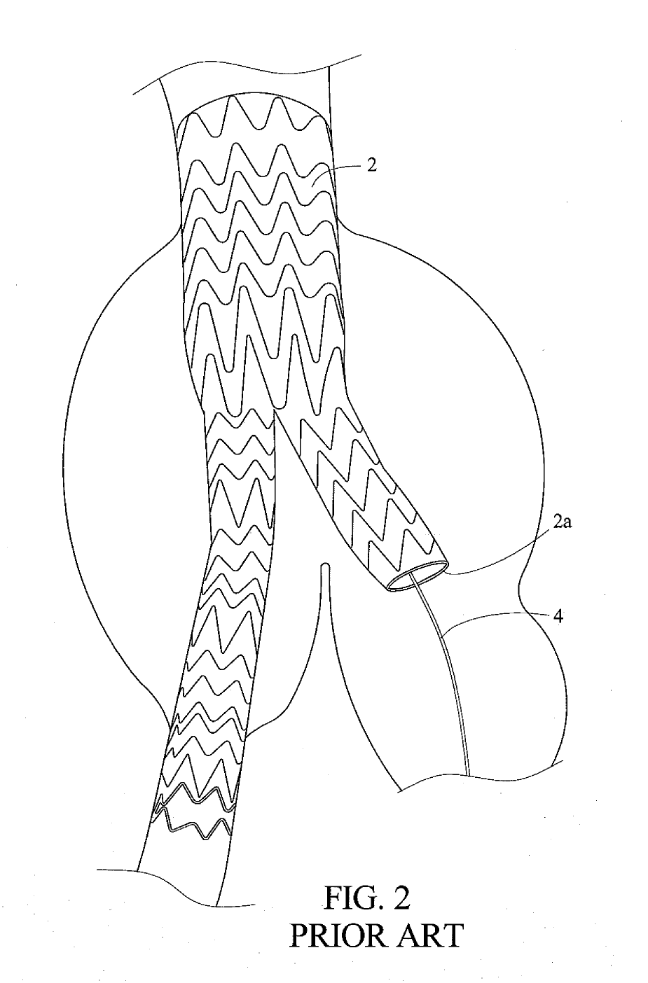

FIG. 3 illustrates a prior art, related to a conventional abdominal aortic stent connecting to a sub-stent.

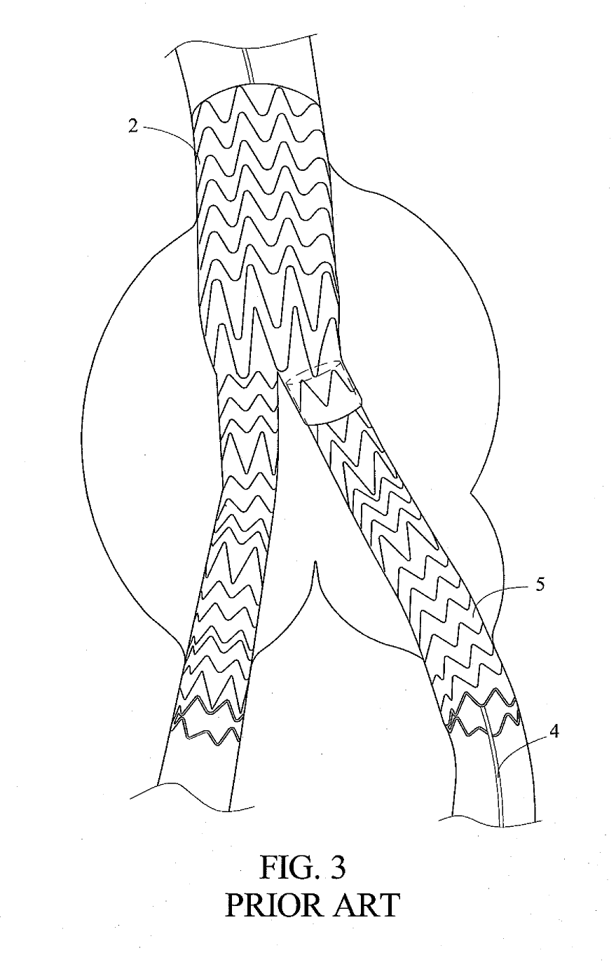

FIG. 4 illustrates a schematic view of one embodiment of the present invention.

FIG. 5 illustrates a schematic view of another embodiment of the present invention.

FIG. 6 illustrates a schematic view of metal wires put into the sub-stents of the present invention.

FIG. 7 illustrates a schematic view of sub-stents with metal wires penetrated through the sub-stents.

FIG. 8 illustrates a schematic view of a further embodiment of the present invention associated with renal artery.

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications which might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

This invention relates to an abdominal aortic stent. More specifically, this invention relates to an abdominal aortic stent structure with a shape which can be easily implanted to reconstruct a vascular flow path. Conventionally, most stents have a Y-shaped structure which is used for the reconstruction on one side of the iliac artery. Before being implanted, a metal wire has to be placed in a preserved path, and the stent can be guided to an appropriate location. However, sometimes if the blood vessel of the patient has a larger angle, it may be hard to cannulate the metal wire to the stent. Furthermore, it may be more difficult to cannulate another metal wire to a branch opening of the stent, which may increase the surgery time and even lead to surgery-related complications.

The present invention is to provide a support structure (i.e. stent) for an abdominal aorta, the stent comprises a first and a second sub-stent, and a front portion of each sub-stent has a cross-sectional area which is half of the abdominal aorta. The cross-sectional area is gradually reduced and at the very end of the sub-stent, it has entered one side of the bilateral femoral arteries, wherein at the very end of the first and second sub-stents, the cross-sectional area of the sub-stents is entirely included in the femoral artery, and then the first and second sub-stents can be fully extended within the vessel to reconstruct the vascular path after the external removable membrane coated on the sub-stents has been removed.

Comparing to the conventional stents stated above, the abdominal aortic stent in the present invention comprises a first and a second sub-stents, each of which constitutes half of the abdominal aortic stent. So, the metal wires can be individually inserted into the arteries to guide the first and second stents to predetermined locations. After removing the membrane thereon, the stent can be fully extended within the vessel to reconstruct the vascular path, which makes the stent more convenient to use. Moreover, it can shorten the surgery time, reduce the surgery-related complications and help the patient recover faster.

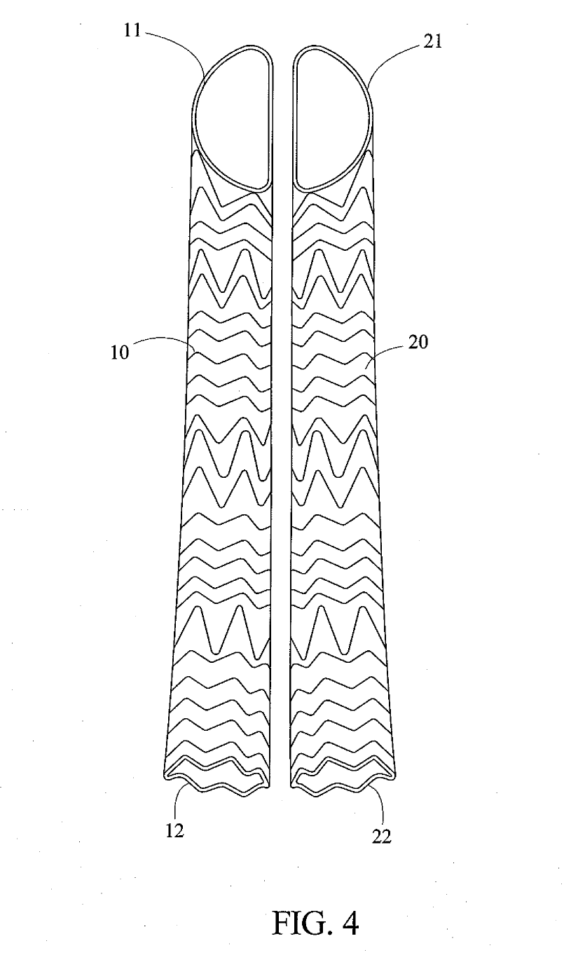

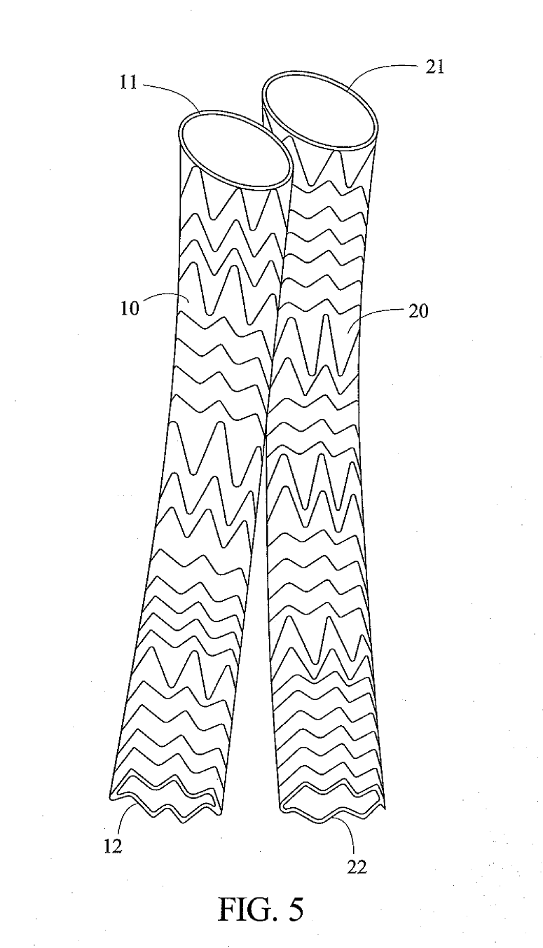

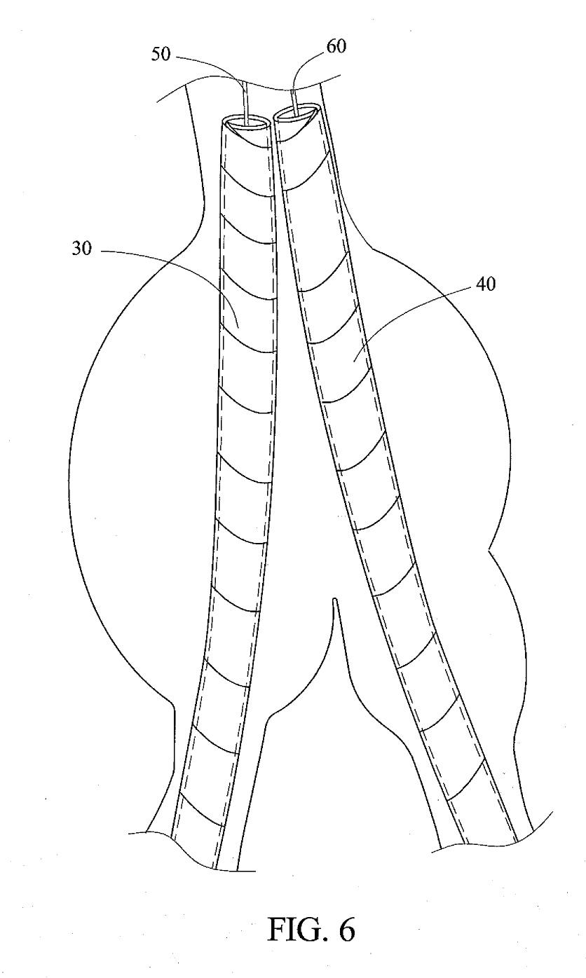

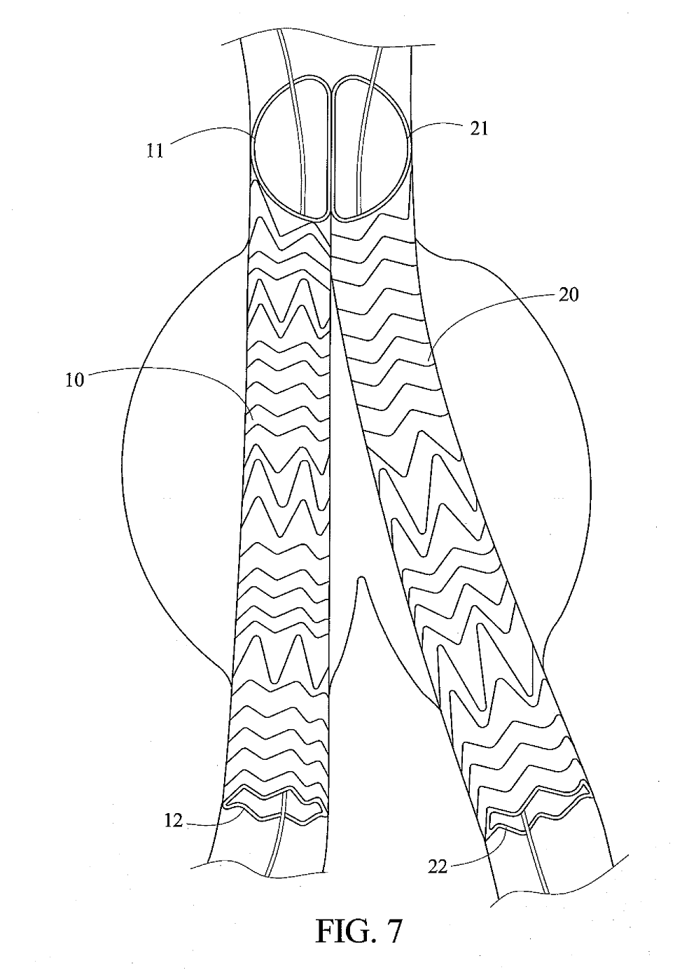

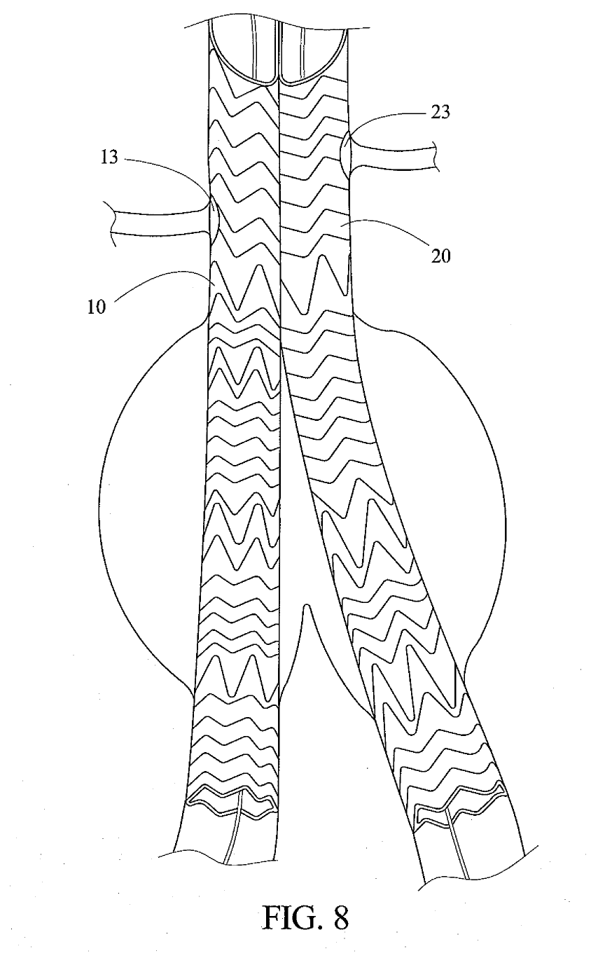

As seen in FIGS. 4 to 8, the present invention is to provide an abdominal aortic stent comprising two almost identical stents: a first sub-stent (10) and a second sub-stent (20), each having a front end (11) and (21), respectively; and the circumference of the front (11, 21) ends of the first and the second stents is half of the circumference of the abdominal aorta, and when the circumference is gradually reduced to a rear end (12, 22) of each sub-stent (10, 20), it has entered one side of the bilateral femoral arteries, wherein the rear ends (12, 22) of the first and second sub-stents (10, 20) with full circumference of the cross-sectional area of the femoral artery are included therein, and the first and second sub-stents (10, 20) are coated with external removable membrane (30, 40) to compress the sub-stents (10, 20) to generate smaller circumferences, and when the membrane (30, 40) has been removed, the sub-stents (10, 20) can be fully extended within the vessel to reconstruct the vascular path.

In one embodiment, the front ends (11, 21) of the first and second sub-stents (10, 20) can be circular or semi-circular.

In another embodiment of the present invention, the abdominal aortic stent comprises the first and second sub-stents (10, 20), so the metal wires (50, 60) can be put through both sides of the femoral artery, and the first and second sub-stents (10, 20) can be guided along the metal wire (50, 60) to reach the predetermined position. When the membrane (30, 40) of the sub-stents (10, 20) is removed, the sub-stents (10, 20) will be fully extended within the vessel to reconstruct the vascular path, which makes the stent more convenient to use. Meanwhile, it can shorten the surgery time, reduce the surgery-related complications and help the patient recover faster.

As to the abdominal aortic aneurysm (junxtarenal abdominal aortic aneurysm) which may affect the renal artery, longer sub-stents (10, 20) with membrane may be selected. Furthermore, small stents used to connect the renal artery can be placed at a first preserved space (13) and a preserved second space (23) in the appropriate place in the sub-stents (10, 20).

Having described the invention by the description and illustrations above, it should be understood that these are exemplary of the invention and are not to be considered as limiting. Accordingly, the invention is not to be considered as limited by the foregoing description, but includes any equivalents.

What is claimed is:

1. An abdominal aortic stent comprising:
a first sub-stent having a front end and a rear end; and
a second sub-stent having a front end and a rear end,
wherein circumference of the front end of each sub-stent is half of circumference of a first predetermined artery and is tapered until circumference of the rear end is fully enclosed by a second predetermined artery; and the sub-stents coated with external removable membrane are fully extended after the external membrane is removed.

2. The abdominal aortic stent of claim 1, wherein shape of the front end of each sub-stent is round.

3. The abdominal aortic stent of claim 1, wherein shape of the front end of each sub-stent is semi-circular.

4. The abdominal aortic stent of claim 1, wherein the first and the second sub-stents are coated with membrane and each sub-stent has a first preserved space and a second preserved space close to the front end for smaller stents to be placed therein.
